SEQUENCE LISTING (1 sequence)

SEQ ID NO: 1 - protein, 13 aa, Artificial Sequence
  Amino acid sequence of Rev peptide of composite nanoparticle
ARRNRRRRWREYC

(19) United States
(12) Patent Application Publication
(10) Pub. No.: US 2012/0328665 A1
(43) Pub. Date: Dec. 27, 2012

(54) COMPOSITE NANOPARTICLE, AND PREPARATION PROCESS AND USE THEREOF

(75) Inventors: Yian Tai, Taipei City (TW); Yat Ming Yung, Hong Kong (HK); Tzu-Hao Wang, New Taipei City (TW); Tran Thi Thanh Ngoc, Taipei City (TW)

(73) Assignee: National Taiwan University of Science and Technology, Taipei City (TW)

(21) Appl. No.: 13/189,092

(22) Filed: Jul. 22, 2011

(30) Foreign Application Priority Data
Jan. 21, 2011 (TW) 100102297

(51) Int. Cl. A61K 9/14; A61K 38/02; A61P 35/00; A61P 31/18; C07K 2/00; C12N 5/09; B82Y 5/00

(52) U.S. Cl. 424/400; 530/300; 428/402; 514/1.1; 435/375; 977/773; 977/915

(57) ABSTRACT

Disclosed herein are a composite nanoparticle and a process for preparing the same. The composite nanoparticle includes Rev peptides chemically bonded to gold. Also disclosed herein are a pharmaceutical composition including a plurality of the aforesaid composite nanoparticles, and a method of inhibiting tumor/cancer cells by virtue of the aforesaid composite nanoparticle.

### CROSS-REFERENCE TO RELATED APPLICATION

This application claims priority of Taiwanese Application No. 100102297, filed on Jan. 21, 2011.

### BACKGROUND OF THE INVENTION

1. Field of the Invention

This application relates to a composite nanoparticle comprising gold and Rev peptides, and a preparation process thereof. This invention also relates to a pharmaceutical composition comprising the composite nanoparticle, and a method for inhibiting tumor/cancer cells by virtue of the composite nanoparticle.

2. Description of the Related Art

Rev peptides have been found to be effective in treating ovarian cancer and other cancers, and AIDS (acquired immunodeficiency syndrome). Specifically, since Rev peptides are capable of binding to nucleophosmin/B23, which is regarded as a cancer marker, and are able to inhibit proliferation of the same, Rev peptides can result in suppression of cancer cell growth and destruction of cancer cells.

U.S. Pat. No. 7,439,223 discloses a method of inhibiting cell growth, which comprises a step of blocking the function of nucleophosmin/B23 within a cell. The aforementioned blocking step is achieved via administration of a nucleophosmin/B23 binding peptide, and is able to induce up-regulation of the transcriptional activity of the tumor suppressor p53. The aforesaid nucleophosmin/B23 binding peptide is a Rev-NLS peptide that has both of a Rev sequence and an NLS (nuclear localization signal) sequence. Once the aforesaid nucleophosmin/B23 binding peptide is introduced into a cell, the same is able to enter the nucleus of the cell, thereby being capable of blocking the function of nucleophosmin/B23. Accordingly, the transcriptional activity of p53 can be up-regulated such that proliferation of cancer cells can be inhibited, and such that the cancer cells can be further exterminated.

Since Rev peptides are unstable molecules, the same are very likely to be decomposed when injected into a living subject. In order to take effect, a great amount of Rev peptides must he injected. However, an excessive amount of Rev peptides may adversely influence a living subject. Thus, even though Rev peptides have been proven to be able to inhibit growth of cancer cells, the same alone are unsuitable to serve as drugs far treating cancer.

Nowadays, gold nanoparticles are used as carriers for drug delivery. Gold nanoparticles are generally synthesized as follows. Ligands capable of reacting with gold ions, such as citrate, oleic acid, oleyl amine, and so forth, are employed to reduce gold ions to gold atoms, and to aggregate the gold atoms so as to form gold nanoparticles. In order to deliver desired drug molecules, the ligands binding to the gold nanoparticles are replaced with ligands capable of binding to both of the desired drug molecules and the gold nanoparticles so that the new ligands can interconnect the desired drag molecules and the gold nanoparticles, or are replaced with the desired drug molecules themselves. However, the aforementioned ligand replacement in drug delivery is laborious and costly. Furthermore, the drug molecules may be detrimentally affected by the ligands binding thereto.

### SUMMARY OF THE INVENTION

According to a first aspect, this invention provides a composite nanoparticle, which comprises gold and Rev peptides chemically bonded to the gold.

According to a second aspect, this invention provides a method for preparing a composite nanoparticle, which comprises contacting a gold ion-containing solution with a Rev peptide-containing aqueous solution. The Rev peptide-containing aqueous solution has a pH value ranging from 1 to 14.

According to a third aspect, this invention provides a pharmaceutical composition comprising a plurality of the aforementioned composite nanaparticles.

According to e fourth aspect, this invention provides a method for inhibiting tumor/cancer cells, which comprises contacting the cells with the aforementioned composite nanoparticle.

### BRIEF DESCRIPTION OF THE DRAWINGS

The above and other objects, features and advantages of this invention will become apparent with reference to the following detailed description and the preferred embodiments taken in conjunction with the accompanying drawings, in which:

FIG. 1 is a transmission electron microscopy photomicrograph showing the shape and the size of the composite nanoparticles prepared according to Example 1 of this invention;

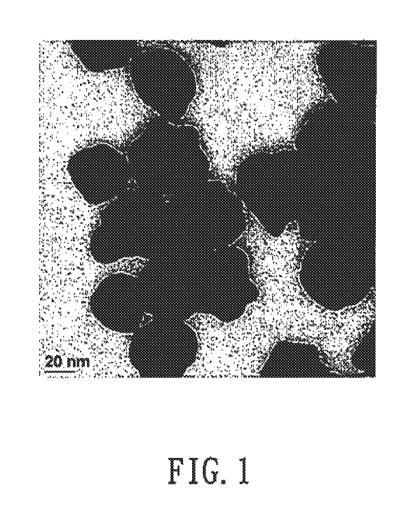

FIG. 2 is an energy dispersion spectrum of the composite nanoparticles prepared according to Example 1 of this invention;

FIG. 3 is an ultraviolet-visible spectrum of the composite nanoparticles prepared according to Example 1 of this invention;

FIG. 4 is a bar diagram showing the cell death percentage of the MOSEC cancer cells, in which the MOSEC cancer cells of the negative control group, the positive control group, and the experimental group were treated respectively with gold nanoparticles (100 μM), Rev peptides (100 μM), and the composite nanoparticles prepared according to Example 1 of this invention (100 μM) for 24 hours;

FIG. 5 is a bar diagram showing the relative cell survival percentage of the MOSEC cancer cells, in which the MOSEC cancer cells of the negative control group, the positive control group, and the experimental group were treated respectively with gold nanoparticles (100 μM), Rev peptides (100 μM), and the composite nanoparticles prepared according to Example 1 of this invention (100 μM) for 48 hours; and

FIG. 6 is a transmission electron microscopy photomicrograph at 8000× magnification, which shows the MOSEC cancer cells cultivated with the composite nanoparticles prepared according to Example 1 of this invention for 24 hours.

### DETAILED DESCRIPTION OF THE PREFERRED EMBODIMENTS

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which this invention belongs. One skilled in the art will recognize many methods and materials similar or equivalent to those described herein, which could be used in the practice of this invention. Indeed, this invention is in no way limited to the methods and materials described.

Inasmuch as Rev peptides are unstable and are unsuitable to be used alone, the applicants have strived to develop a way to deliver Rev peptides into a living subject. The applicants have combined Rev peptides with gold by virtue of a redox reaction to form a composite nanoparticle. The aforesaid composite nanoparticle has been found to be stable in a living subject such that the possibility of the decomposition of the Rev peptides thereof is low. Accordingly, the Rev peptides of the aforesaid composite nanoparticle can efficiently bind to nucleophosmin/B23, thereby being able to effectively inhibit cancer cells.

Therefore, this invention provides a composite nanoparticle, which comprises gold and Rev peptides chemically bonded to the gold. Preferably, the composite nanoparticle has a core composed of the gold and a shall composed of the Rev peptides.

According to this invention, the composite nanoparticle has a particle size ranging from 1 nm to 100 nm. Preferably, the composite nanoparticle has a particle size ranging from 1 nm to 50 nm. More preferably, the composite nanoparticle has a particle size ranging from 20 nm to 40 nm.

According to this invention, the composite nanoparticle has a polygonal shape or a sphere-like shape.

In a preferred embodiment of this invention, the Rev peptides of the composite nanoparticle have an amino acid sequence as shown in SEQ ID NO:1.

The composite nanoparticle according to this invention is prepared by virtue of a method, which comprises contacting a gold ion-containing solution with a Rev peptide-containing aqueous solution. The Rev peptide-containing aqueous solution has a pH value ranging from 1 to 14. Preferably, the Rev peptide-containing aqueous solution has a pH value ranging from 5 to 13. More preferably, the Rev peptide-containing aqueous solution has a pH value ranging from 10 to 12.

According to this invention, the contacting of the gold ion-containing solution with the Rev peptide-containing aqueous solution is conducted at a temperature ranging from 5° C. to 100° C. Preferably, the contacting of the gold ion-containing solution with the Rev peptide-containing aqueous solution is conducted at a temperature ranging from 15° C. to 30° C.

According to this invention, a mixture obtained by contacting the geld ion-containing solution with the Rev peptide-containing aqueous solution may be further subjected to a centrifugation treatment. The resultant supernatant contains a plurality of the composite nanaoparticles which have a particle size ranging from 1 nm to 50 nm. Optionally, the composite nanoparticles may be further isolated from the supernatant by virtue of reduced pressure distillation or a drying process.

According to this invention, the gold ion-containing solution can be made from any material that is able to provide gold ions capable of reacting with Rev peptides. Preferably, the gold ion-containing solution may be made from a material selected from the group consisting of chloroauric acid and gold sulfite.

According to this invention, the Rev peptide-containing aqueous solution includes a solvent that can be made from any material capable of dissolving Rev peptides. Preferably, the solvent may be made from a material selected from the group consisting of sodium hydroxide and sodium bisulfate.

The applicants have conducted in vitro experiments to investigate the effect of the composite nanoparticle according to this invention on cancer cells. The experimental results show that the composite nanoparticle according to this invention la effective in inhibiting growth of cancer cells. Accordingly, this invention provides a method for inhibiting tumor/cancer cells, which comprises contacting the cells with the composite nanoparticle as described above.

Since U.S. Pat. No. 7,439,223 has disclosed that Rev peptides can inhibit growth of cancer/tumor cells in vivo, the composite nanoparticle according to this invention is expected to be useful in treatment of cancers. Accordingly, this invention provides a pharmaceutical composition comprising a plurality of the composite nanoparticles as described above. Preferably, the pharmaceutical composition is in the form of a suspension that contains a suspending medium and the composite nanoparticles suspended in the suspending medium. The suspending medium may be a buffer solution suitable for human consumption.

This invention will be farther described by way of the following examples. However, it should be understood that the following examples are solely intended for the purpose of illustration and should not be construed as limiting the invention in practice.

### EXAMPLES

### Experimental Materials:

1. Chloroauric acid (HAuCl₄) was purchased from Acros Organics.
2. Rev peptides were synthesized by Kelowna International Scientific Inc. (Taiwan).
3. Sodium hydroxide (NaOH) was purchased from Acros Organics.
4. Cancer cells:

Cancer cells used in the examples below are MOSEC (mouse ovarian surface epithelial cell) cancer cells which were provided by Dr. C. L. Chang from Department of Gynaecology at Mackay Memorial Hospital (C. L. Chang et al. (2007), Cancer Res., 67:10047-10057). MOSEC cancer cells were placed in a Petri dish containing RPMI 1640 medium (Invitrogen) supplemented with 10% fetal bovine serum (FBS), 100 U/mL penicillin, and 100 U/mL streptomycin, followed by cultivation in an incubator with culture conditions set at 37° C. and 5% CO₂.

### Example 1

Preparation of Composite Nanoparticle According to this Invention

A proper amount of HAuCl₄ was dissolved in 2 mL of deionized water so as to form a gold ion-containing solution having a gold ion concentration of 0.001 M. 1 mL of the 5 mM Rev peptide solution was mixed with 1 mL of 0.01 M NaOH aqueous solution, thereby forming a Rev peptide-containing aqueous solution having a Rev peptide concentration of 2.5 mM. The gold ion-containing solution was added dropwise into the Rev peptide-containing aqueous solution, followed by stirring at 25° C. and 500 rpm for 4 hours. The resultant mixture was left standing for 1 day so that the redox reaction between gold ions and Rev peptides could be complete. Subsequently, the mixture left standing for 1 day was subjected to centrifugation at 24000 rpm for 30 minutes. The resultant supernatant contained the composite nanoparticles according to this invention, and served as a sample solution.

### Example 2

### Analysis of Composite Nanoparticle According to the Invention

The composite nanoparticles prepared in Example 1 were analysed by transmission electron microscopy (TEM), X-ray photoelectron spectroscopy (XPS), ultraviolet-visible-near infrared (UV-VIS-NIR) spectroscopy, etc.

### A. Observation by TEM

The composite nanoparticles prepared in Example 1 were observed under a TEM device (Philips Tecnai F20 G2). Specifically, several drops of the sample solution prepared in Example 1, which contained the composite nanoparticles according to this invention, were placed onto a TEM copper grid, followed by drying under vacuum. Therefore, a TEM sample was prepared. Afterward, the TEM sample was observed under the TEM device. Selected area electron diffraction (SAED) patterns of the TEM sample were obtained using the TEM device so as to investigate the crystal structure of the composite nanoparticles according to this invention. Furthermore, the energy dispersion spectrum of the TEM sample was acquired via an energy dispersion spectrometer with which the TEM device was equipped.

### Results:

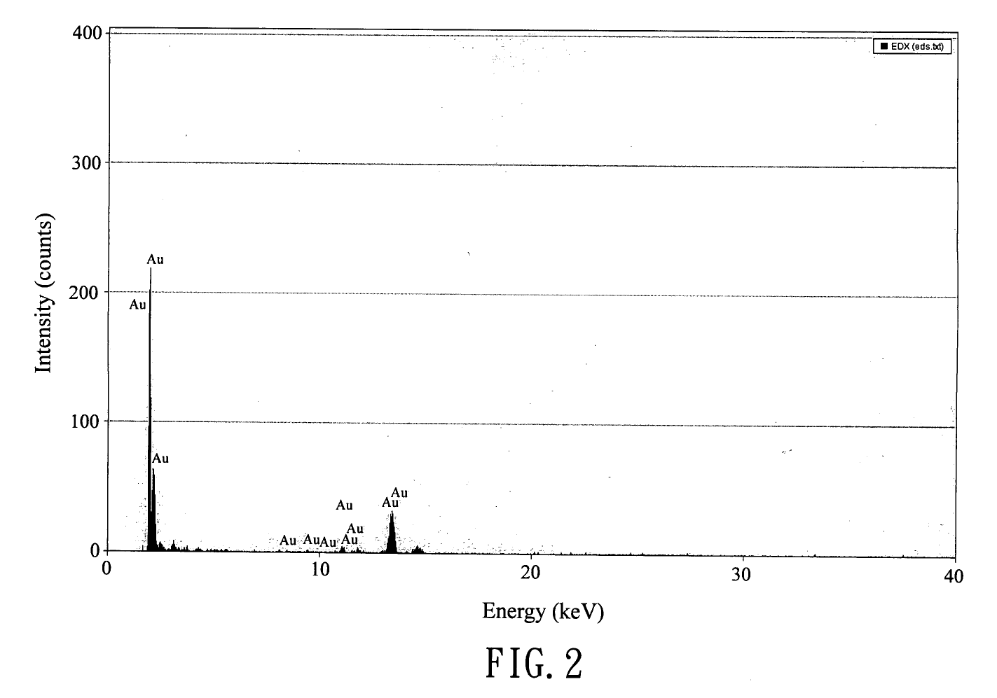

Referring to FIG, 1, the composite nanoparticles have a particle else of about 30 nm and a polygonal shape. Referring to FIG. 2, the composite nanoparticles are shown to contain gold. The SAED patterns of the TEM sample reveal that the composite nanoparticles have the polycrystalline structure of gold, thereby indicating that Au³⁺ ions have been reduced to Au atoms. The reduction of Au³⁺ ions to Au atoms is likely to-be related to a carboxyl group (—COOH), an amine group (—NH₂), or a thiol group (—SH) of Rev peptides.

### B. Composition Analysis by XPS

The sample solution prepared in Example 1, which contained the composite nanoparticles according to this invention, was analysed using an X-ray photoelectron spectrometer (VG ESCA Scientific, Sigma Theta probe, X-ray source: monochromatic A1 Kα (1486.7 eV)) so as to examine the composition thereof.

### Results:

The resultant XPS spectrum shows signals of carbon (C), nitrogen (N), sulfur (S), and oxygen (O). The signals of C, N, S, and O are attributed to the Rev peptides of the composite nanoparticles according to this invention.

### C. Absorbance Measurement by UV-VIS-NIR Spectroscopy

Absorbance regarding the sample solution prepared in Example 1, which contained the composite nanoparticles according to this invention, was measured using a UV-VIS-NIR spectrophotometer (Jas.co V-670).

### Results:

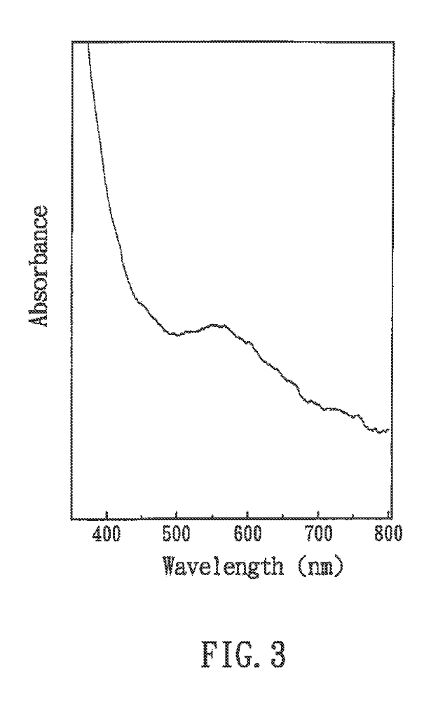

Referring to FIG. 3, a surface plasmon resonance (SPR) band was observed at about 550 nm. The aforementioned SPR band at 528 nm is an absorption band of gold particles having a particle size of several tens of nanometers.

### Example 3

Effect of Composite Nanoparticle According to this Invention on Cancer Cells

In vitro cytotoxicity tests were conducted to investigate the effect of the composite nanoparticle according to this invention on cancer cells.

MOSEC cancer cells were divided into three groups including a positive control group, a negative control group, and en experimental group. The MOSEC cancer cells in each group were placed in a 96-well plate containing RFMI 1640 medium supplemented with 10% FBS and antibiotics (each well contained 200 μL of the aforesaid RPMI 1640 medium) at a concentration of 2.5×10⁴ cell/well, followed by cultivation in an incubator (37° C., 5% CO₂) for 24 hours. The old medium was replaced with the fresh serum-free RFMI 1640 medium. Subsequently, 20 μL of the sample solution prepared in Example 1, which contained the composite nanoparticles according to this invention, was subjected to a filter sterilization treatment using a sieve with a pore size of 0.22 μm, followed by dilution with a sterile buffer solution. The resultant solution had a composite nanoparticle concentration of 100 μM, served as a test solution, and was added into the culture of the MOSEC cancer cells in the experimental group. The test solution containing only Rev peptides (the Rev peptide concentration thereof was 100 μM) and the test solution containing only gold nanoparticles (the gold nanoparticle concentration thereof was 100 μM) were prepared using a sterile buffer solution, and were respectively added into the cultures of the MOSEC cancer cells in the positive control group and the negative control group. The MOSEC cancer cells in each group were cultivated in an incubator (37° C., 5% CO₂). The MOSEC cancer cells in each group were harvested at the 24^{th} hour of the cultivation to perform the experiment in the following section A, and were collected at the 48^{th} hour of the cultivation to conduct the experiment in the following section B, and the MOSEC cancer cells in the experimental group were harvested at the 24^{th} hour of the cultivation to perform the experiment in the following section C.

### A. Lactate Dehydrogenase (LDH) Assay

100 μL of the cultured medium of the MOSEC cancer cells in each group was placed in a new 96-well plate. Afterward, the activity of lactate dehydrogenase was measured using cytotoxicity detection kit (Cat. No. 11644793001, Roche Applied Science, Indianapolis, Ind., USA) according to the manufacturer's instructions. Consequently, the cell death percentage (%) regarding the MOSEC cancer cells in each group could fee calculated.

### Results:

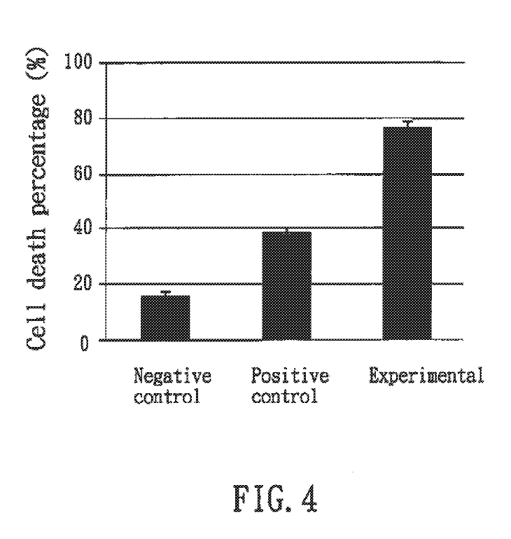

Referring to FIG. 4, the cell death percentage regarding the MOSEC cancer cells in each of the positive control group and the experimental group is higher than that regarding the MOSEC cancer cells in the negative control group. In particular, the cell death percentage regarding the MOSEC cancer cells in the experimental group is higher than that regarding the MOSEC cancer cells in the positive control group. The results manifest that: the composite nanoparticles according to this invention are cytotoxic to cancer cells, and are even mere effective than Rev peptides alone.

B. 3-[4,5-Dimethylthiazol-2-yl]-2,5-diphenyltetrazolium bromide (MTT) Assay

MTT (5 mg/mL, 25 μL) was added into the culture of the MOSEC cancer cells in each group, followed by cultivation for 4 hours. The liquid in each well was removed. 100 μL of DMSO was added into each well, followed by mixing uniformly. Absorbance of the resultant mixture at 570 nm (OD₅₇₀) was measured using an ELISA reader scanning multi-well spectrophotometer (Perkin Elmer VICTOR 2, GMI, Minnesota, USA).

The relative cell survival percentage (%) can be calculated by substituting the absorbance into the following formulas

A=(B/C)×100 (1)

where A=relative cell survival percentage
B=OD₅₇₀ of a respective one of the experimental group, positive control group, and the negative control group
C=OD₅₇₀ of the negative control group

### Results:

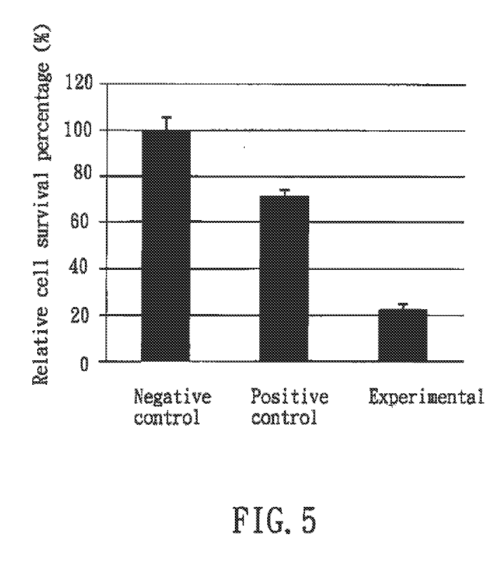

Referring to FIG. 5, the relative cell survival percentage regarding the MOSEC cancer cells in each of the positive control group and the experimental group is lower than that regarding the MOSEC cancer cells in the negative control group. Particularly, the relative cell survival percentage regarding the MOSEC cancer cells in the experimental group is lower than that regarding the MOSEC cancer cells in the positive control group. The results reveal that the composite nanoparticles according to this invention are more effective in inhibiting growth of cancer cells than Rev peptides alone.

### C. Observation by TEM

The culture of the MOSEC cancer cells in the experimental group was collected, followed by fixation with 100 μL of a phosphate buffer solution containing 3% glutaraldehyde. Post-fixation was performed using osmium tetroxide for 24 hours. Sequentially, the fixed cells were subjected to dehydration, using ethanol and were embedded in Epon 812. A microtome was used to slice the resultant specimen such that an ultrathin section having a thickness of 80 nm was obtained. The ultrathin section was stained with 4% uranyl acetate and lead citrate, followed by observation under a TEM device (Hitachi H-7500).

### Results:

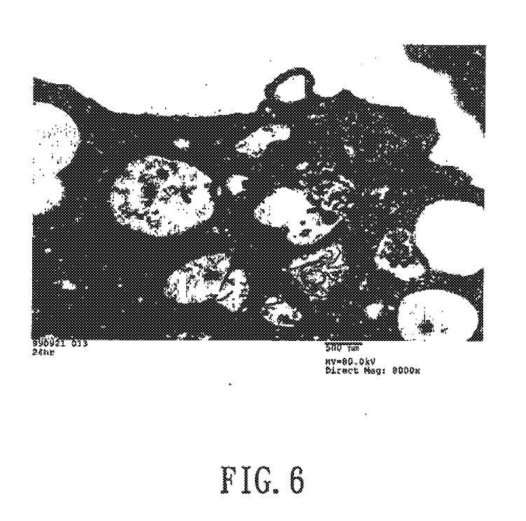

Referring to FIG. 6, it is shown that the composite nanoparticles according to this invention are able to enter the cells cultivated with the same for 24 hours.

All patents and literature references cited in the present specification as well as the references described therein, are hereby incorporated by reference in their entirety. In case of conflict, the present description, including definitions, will prevail.

While the invention has been described with reference to the above specific embodiments, it is apparent that numerous modifications and variations can be made without departing from the scope and spirit of this invention. It is therefore intended that this invention be limited only as Indicated by the appended claims.

What is claimed is:

1. A composite nanoparticle comprising;
gold; and
Rev peptides chemically bonded to the gold.

2. The composite nanoparticle of claim 1, which has a particle size ranging from 1 nm to 100 nm.

3. The composite nanoparticle of claim 2, which has a particle size ranging from 1 nm to 50 nm.

4. A method for preparing a composite nanoparticle, comprising:
contacting a gold ion-containing solution with a Rev peptide-containing aqueous solution;
wherein the Rev peptide-containing aqueous solution has a pH value ranging from 1 to 14.

5. The method of claim 4, wherein the gold ion-containing solution is made from a material selected from the group consisting of chloroauric acid and gold sulfite.

6. The method of claim 4, wherein the Rev peptide-containing aqueous solution includes a solvent that is made from a material selected from the group consisting of sodium hydroxide and sodium bisulfate.

7. A pharmaceutical composition comprising a plurality of composite nanoparticles as claimed in claim 1.

8. The pharmaceutical composition of claim 7, wherein the composite nanoparticles have a particle size ranging from 1 nm to 100 nm.

9. The pharmaceutical composition of claim 8, wherein the composite nanoparticles have a particle size ranging from 1 nm to 50 nm.

10. The pharmaceutical composition of claim 7, which is in the form of a suspension.

11. A method for inhibiting tumor/cancer cells, comprising contacting the cells with a composite nanoparticle as claimed in claim 1.
